(19) United States
(12) Patent Application Publication
(10) Pub. No.: US 2026/0306879 A1
(43) Pub. Date: Oct. 8, 2026

(54) PARTICLE THERAPY SYSTEM AND METHOD FOR OPERATING THE SAME

(71) Applicant: EBG MedAustron GmbH, Wiener Neustadt (AT)

(72) Inventors: Alexander WASTL, Ferlach (AT); Sebastian NOWAK, Feistritz am Wechsel (AT)

(73) Assignee: EBG MedAustron GmbH, Wiener Neustadt (AT)

(21) Appl. No.: 19/483,111

(22) PCT Filed: May 7, 2024
(86) PCT No.: PCT/AT2024/060193

§ 371 Date: Nov. 10, 2025

(30) Foreign Application Priority Data
May 11, 2023 (AT) A503692023

(51) Int. Cl. A61N 5/10

(52) U.S. Cl. CPC A61N 5/1077; A61N 2005/1085

(57) ABSTRACT

A particle therapy system includes a hadron therapy system, comprising a particle source, a pre-acceleration device connected to the particle source for accelerating the particles coming from the particle source. A synchrotron with a circulating particle path has an injection site and an extraction site. A transfer beam guide leads from the pre-acceleration device to the injection site for injecting the particles accelerated by the pre-acceleration device into the particle path of the synchrotron. The pre-acceleration device is arranged above or below the path plane (B) of the synchrotron, wherein the pre-acceleration device vertically overlaps a first path section of the circulating particle path of the synchrotron.

### BACKGROUND

The disclosure relates to a particle therapy system, in particular a hadron therapy system. The disclosure relates to a hospital and a method for operating a particle therapy system.

State-of-the-art particle therapy systems based on a synchrotron require a large amount of space and can therefore only be installed in buildings specifically designed for this purpose. The size of such systems is determined by the individual components and their spatial arrangement in relation to each other. The individual components, in particular the ion source, the pre-accelerator, and the synchrotron, are arranged next to each other on one level. The necessary infrastructure for power supply and cooling, as well as other components for beam guidance, in particular the beam guidance from the extraction site in the synchrotron to the treatment unit, are also arranged on this level.

The space required for such an arrangement prevents the installation of such particle therapy systems, especially hadron therapy systems such as proton therapy systems, directly in hospitals.

In contrast to conventional photon radiation therapy, particles, in particular protons, can penetrate much deeper into human tissue (up to about 30 cm). This means that hadron therapy can also be used to irradiate deep-seated tumors effectively and with few side effects.

### OVERVIEW

The object of the present disclosure is to overcome the disadvantages of the prior art and to provide a particle therapy system and a method for operating such a system, which can significantly reduce the space required. This should also enable the installation of such a particle therapy system in smaller rooms, in particular in hospital premises. The system should be characterized by its compactness and significantly reduced footprint.

This object is achieved by a particle therapy system according to claim 1.

The particle therapy system according to the disclosure, in particular a hadron therapy system, comprises
a particle source,
a pre-acceleration device connected to the particle source for accelerating the particles coming from the particle source,
a synchrotron with a circulating particle path having an injection site and an extraction site,
a transfer beam guide leading from the pre-acceleration device to the injection site for injecting the particles accelerated by the pre-acceleration device into the particle path of the synchrotron.

The particle therapy system is characterized in that the pre-acceleration device is arranged above or below the path plane of the synchrotron (main accelerator), whereby the pre-acceleration device overlaps-at least partially-vertically with a first path section of the circulating particle path of the synchrotron.

This arrangement places the space-consuming pre-accelerator, which usually comprises a linear accelerator with a length of more than 1 m, usually several meters, in a plane outside the plane of the synchrotron's trajectory, thereby significantly reducing the footprint of the system. The vertically overlapping arrangement of the pre-accelerator and a first section of the synchrotron's circulating particle path also means that the space enclosed by the synchrotron ring is not taken up by the pre-accelerator, thus providing space for other components such as the power supply device, vacuum generation device, cooling device, and control device.

The particle source, particularly in the form of an ion source, provides the particles intended for irradiation. These are preferably hadrons, particularly protons.

The particles coming from the ion source are accelerated in the pre-acceleration device (e.g., to an energy of 7 MeV) before being injected into the synchrotron. The pre-acceleration device may comprise several components which, in addition to acceleration, also effect beam shaping/bundling. Preferably, the pre-acceleration device comprises at least one linear accelerator (Linac), preferably in the form of a quadrupole radio frequency accelerator (RFQ Linac).

A synchrotron is a particle accelerator in the form of a ring accelerator. Charged elementary particles or ions can be accelerated to very high speeds in the accelerator, giving them very high kinetic energies. The energy or energy distribution of the particles, which are then guided via the extraction site towards the treatment unit, is preferably variably adjustable in a synchrotron.

Vertically overlapping means that the pre-acceleration device and the particle path of the synchrotron are superimposed vertically, i.e., in the vertical direction. In other words, when viewed from above, one of these components (e.g., the pre-acceleration device) obscures the other component (particle path of the synchrotron). It is possible to arrange the pre-acceleration device above the particle path or below the particle path. As already mentioned above, this reduces the required footprint. It is therefore preferable for the pre-acceleration device to be arranged vertically above or below the first path section.

It is preferred that the pre-acceleration device overlaps vertically with the first path section along at least 50%, preferably at least 75%, and particularly preferably at least 100% of its longitudinal extent or the longitudinal extent of its beam path. The overlap in the specified range is preferably continuous, i.e., without interruption.

If the first path section is an (exclusively) linear path section, it is preferred that the ratio between the length of the beam path within the pre-acceleration device or within a linear accelerator of the pre-acceleration device and the length of the first (linear) path section is between 0.7 and 1.3, preferably between 0.85 and 1.15. This shows that the coordinated dimensions can lead to maximum space savings.

The particle path of the synchrotron is formed by a vacuum-pressurized tube in which the particles are kept in the beam path by appropriate beam guidance components, in particular magnets.

A preferred embodiment is characterized in that the extraction site is arranged in the first path section and the pre-acceleration device vertically overlaps the extraction site. In this way, a particularly compact design can be achieved, since the components for pre-acceleration and those for extraction (including the extraction beam guidance) can be accommodated in a very small space, thus making optimum use of the available space.

A preferred embodiment is characterized in that the particle source at least partially vertically overlaps the first section of the circulating particle path of the synchrotron. This measure means that the particle or ion source also contributes little or nothing to the footprint of the system.

A preferred embodiment is characterized in that the first path section is a linear path section and the pre-acceleration device comprises a linear accelerator, wherein the beam path of the first path section and the beam path of the linear accelerator are essentially parallel to each other or have a deviation from a parallel alignment of no more than 20°. In other words, the relevant (linear) beam paths are parallel to each other or inclined at an angle of no more than 20° to each other. This measure ensures the greatest possible overlap.

A preferred embodiment is characterized in that the transfer beam path between the pre-acceleration device and the injection site overcomes a difference in height.

A preferred embodiment is characterized in that the transfer beam guide extends laterally beyond the outer contour of the circulating particle path of the synchrotron. This makes it possible, on the one hand, to accommodate the pre-accelerator device completely on one side of the synchrotron and, on the other hand, to inject the pre-accelerated particles into the synchrotron in a direction that is horizontal or has a horizontal directional component. The latter further reduces the space requirement in the horizontal direction because no deflection magnets need to be provided in the region of the injection site.

A preferred embodiment is characterized in that the transfer beam guide has a first path section, which is used to overcome the height difference between the pre-acceleration device and the path plane of the synchrotron, and a second path section, which runs between the first path section and the injection site in the path plane of the synchrotron.

A preferred embodiment is characterized in that the transfer beam guide opens into the injection site with a horizontal directional component, preferably from a region outside the region enclosed by the particle path.

A preferred embodiment is characterized in that the injection site is arranged in a second, preferably linear, path section of the synchrotron, wherein the beam direction defined by the second path section and the beam direction defined by the first path section are inclined to each other by an angle which is preferably at least 60° and no more than 120°, more preferably 90°. Due to this geometry, the particles do not need to be deflected further in the transfer beam guide, which can therefore be designed to be significantly shorter and thus more space-saving.

A preferred embodiment is characterized in that the first path section, which is a linear path section, transitions directly into an arcuate path section and the arcuate path section transitions directly into the second path section, which is a linear path section, wherein the arcuate path section is preferably a 90° arc. This measure allows the transfer beam guide to be guided in a particularly space-saving manner.

A preferred embodiment is characterized in that an electromagnetic shield, preferably in the form of a plate, foil, grid, or mesh, made of electrically conductive material is arranged between the pre-accelerator device and the first path section. This efficiently prevents adverse mutual interference between the superimposed components, in particular due to stray fields.

A preferred embodiment is characterized in that the pre-accelerator device is at least partially supported by the same frame that also supports the synchrotron. This saves on additional components, allowing the system to be implemented in a more material-and cost-efficient manner.

A preferred embodiment is characterized in that the pre-accelerator device and/or the particle source is supported by at least one, preferably at least two, support strut(s) extending from bottom to top, at least one support strut being arranged on the outside of an arcuate path section of the synchrotron. This allows the corner region (which would otherwise usually be un-used) to be used to support the pre-acceleration device/particle source, further increasing the compactness of the design.

A preferred embodiment is characterized in that the particle system comprises an extraction beam guide extending from the extraction site and leading to a treatment unit, wherein the ex-traction beam guide preferably has an arcuate path section, preferably in the form of a 90° arc. At the treatment unit itself, it is preferable for the patient to be immobilized and treated in a seated position on a movable element. The patient and/or the particle beam are aligned so the latter it hits the area to be treated.

A preferred embodiment is characterized in that the projection of the transfer beam guide into the path plane of the synchrotron lies-at least in partially-between the particle path of the synchrotron and the extraction beam guide. This allows the region between the course of the extraction beam guide and the synchrotron, which would otherwise remain free, to be used.

A preferred embodiment is characterized in that a contour drawn in a horizontal plane around the arrangement of synchrotron, particle source, pre-acceleration device, extraction beam guide, and treatment unit encloses an area of no more than 100 m², preferably no more than 85 m². This allows particle therapy systems according to the disclosure to also be installed in existing hospitals.

A preferred embodiment is characterized in that the particle therapy system comprises a power supply device, a vacuum generation device, a cooling device, and a control device, wherein at least one, preferably at least two, of these components is/are arranged above and/or below the synchrotron and/or within the circulating particle path of the synchrotron. This allows the required floor space to be further reduced.

The object is also achieved with a particle therapy system, in particular a hadron therapy system, preferably according to one of the preceding claims, comprising
a particle source,
a pre-acceleration device connected to the particle source for accelerating the particles coming from the particle source,
a synchrotron with a circulating particle path having an injection site and an extraction site,
a transfer beam guide leading from the pre-acceleration device to the injection site for injecting the particles accelerated by the pre-acceleration device into the particle path of the synchrotron,
   characterized in that the particle therapy system has a control device with an operating mode for feeding the energy stored in the magnets-provided for guiding or deflecting the particles back into an electrical energy storage device (e.g., buffer storage, battery, etc.) or into the power grid.

This measure also leads (in addition to energy savings) to a system being able to be designed more compactly. Feeding back of the energy stored in the magnetic fields allows the cooling devices—which would otherwise have to dissipate all of the energy converted into heat—to be smaller in size and therefore contribute less to the footprint of the system.

The object is also achieved with a particle therapy system, in particular a hadron therapy system, preferably according to one of the preceding claims, comprising
a particle source,
a pre-acceleration device connected to the particle source for accelerating the particles coming from the particle source,
a synchrotron with a circulating particle path having an injection site and an extraction site,
a transfer beam guide leading from the pre-acceleration device to the injection site for injecting the particles accelerated by the pre-acceleration device into the particle path of the synchrotron,
   characterized in that the particle therapy system comprises a control device with an operating mode for decelerating the particles in the synchrotron, wherein the operating mode is configured to reduce the velocity of the particles by controlling at least one cavity of an acceleration section of the synchrotron, by generating a force acting on the particles in the at least one cavity in the opposite direction to their direction of movement. This can be achieved by applying the high-frequency coil(s) of the cavity 26 in such a way that the high-frequency electromagnetic waves accelerate the particles in the opposite direction to their direction of movement (i.e., actively decelerate them).

The active reduction of particle energy in the synchrotron (main accelerator) significantly reduces radiation exposure, which also allows radiation protection measures to be reduced and consequently also reduces the space required.

The disclosure also relates to a hospital in which a particle therapy system according to the disclosure is installed.

The disclosure also relates to a method for operating a particle therapy system according to the disclosure, in which method the particles coming from the particle source are accelerated by the pre-acceleration device and are injected into the synchrotron by means of the transfer beam guide. Here, the particles are guided from a first plane (plane of the pre-acceleration device) into the plane of the synchrotron, whereby the particles overcome a vertical height difference (upward or downward).

The object is also achieved by a method for operating a particle therapy system, preferably according to the claims, wherein the particle therapy system comprises
a particle source,
a pre-acceleration device connected to the particle source for accelerating the particles coming from the particle source,
a synchrotron with a circulating particle path having an injection site and an extraction site,
a transfer beam guide leading from the pre-acceleration device to the injection site for injecting the particles accelerated by the pre-acceleration device into the particle path of the synchrotron
   characterized in that, in one operating mode of the particle therapy system, the energy stored in the magnets—provided for guiding or deflecting the particles—is fed into an electrical energy storage device or into the power grid.

The object is also achieved by a method for operating a particle therapy system, preferably according to the claims, wherein the particle therapy system comprises
a particle source,
a pre-acceleration device connected to the particle source for accelerating the particles coming from the particle source,
a synchrotron with a circulating particle path having an injection site and an extraction site,
a transfer beam guide leading from the pre-acceleration device to the injection site for injecting the particles accelerated by the pre-acceleration device into the particle path of the synchrotron
   characterized in that, in one operating mode, the particles are decelerated in the synchrotron by controlling at least one cavity of an acceleration section of the synchrotron in order to reduce the velocity of the particles by generating a force acting on the particles through the at least one cavity in a direction opposite to their direction of movement.

The advantages described above in connection with the particle therapy system also apply equally to the methods.

### BRIEF DESCRIPTION OF THE FIGURES

For a better understanding of the disclosure, it is explained in more detail with reference to the following figures.

Each of the following figures shows a highly simplified, schematic representation:

FIG. 1 a preferred embodiment of a particle therapy system according to the disclosure, viewed from above;

FIG. 2 a particle therapy system in perspective view;

FIG. 3 a section of a particle therapy system, viewed from the side;

FIG. 4 an alternative embodiment of a particle therapy system according to the disclosure viewed from the side; and

FIG. 5 a schematic representation of a hospital with a particle therapy system.

### DETAILED DESCRIPTION OF THE EMBODIMENTS

First, it should be noted that in the various embodiments described, identical parts are assigned identical reference numerals or identical component designations, whereby the disclosures contained in the entire description can be applied mutatis mutandis to identical parts with identical reference numerals or identical component designations. The positional information chosen in the description, such as above, below, side, etc., also refers to the figure directly described and depicted, and this positional information can be transferred to the new position in the same way if the position changes.

The embodiment examples show possible embodiment variants, whereby it should be noted at this point that the disclosure is not limited to the specifically illustrated embodiment variants, but rather that various combinations of the individual embodiment variants are also possible, and that this possibility of variation is within the skill of a person skilled in the art based on the teaching of the technical action of the present disclosure.

The scope of protection is determined by the claims. However, the description and drawings are to be used to interpret the claims. Individual features or combinations of features from the various embodiments shown and described may represent independent inventive solutions.

The object underlying the independent inventive solutions can be found in the description.

All information on value ranges in the present description is to be understood as including any and all sub-ranges thereof, e.g., the specification 1 to 10 is to be understood as including all sub-ranges, starting from the lower limit 1 and the upper limit 10, i.e., all sub-ranges start with a lower limit of 1 or greater and end with an upper limit of 10 or less, e.g., 1 to 1.7, or 3.2 to 8.1, or 5.5 to 10.

For the sake of clarity, it should be noted that, for a better understanding of the structure, some elements have been shown out of scale and/or enlarged and/or reduced in size.

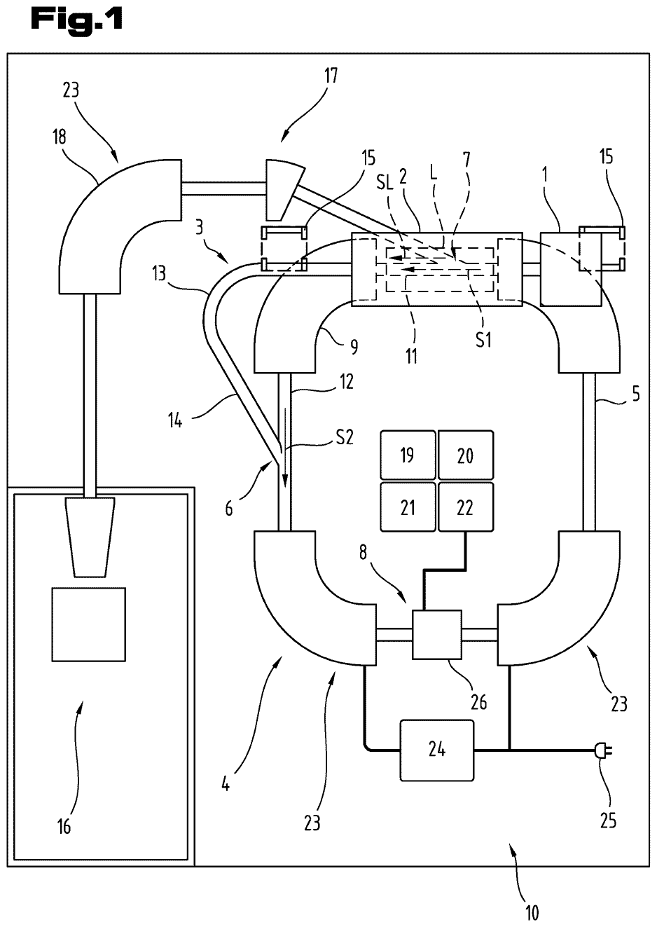

FIG. 1 shows a preferred embodiment of a particle therapy system 10 according to the disclosure, in particular a hadron therapy system. A pre-acceleration device 2 connected to the particle source 1 serves to accelerate the particles coming from the particle source 1. A transfer beam guide 3 leads from the pre-acceleration device 2 to the injection site 6 of the circulating particle path 5 of the synchrotron 4. There, the particles accelerated by the pre-acceleration device 2 are injected into the particle path 5 of the synchrotron 4.

The particles are further accelerated in the synchrotron 4. For this purpose, the synchrotron 4 has at least one acceleration section 8, which comprises a cavity 26 (hollow resonator). In the cavity, the particles are accelerated with the aid of electric fields by exposing them to standing electromagnetic waves of high frequency.

The particles, which have been brought to the desired kinetic energy, are decoupled from the synchrotron at an extraction site 7 and guided to a treatment unit 16 by means of an extraction beam guide 17. The treatment unit is arranged in a room separate from the synchrotron with special radiation-absorbing walls.

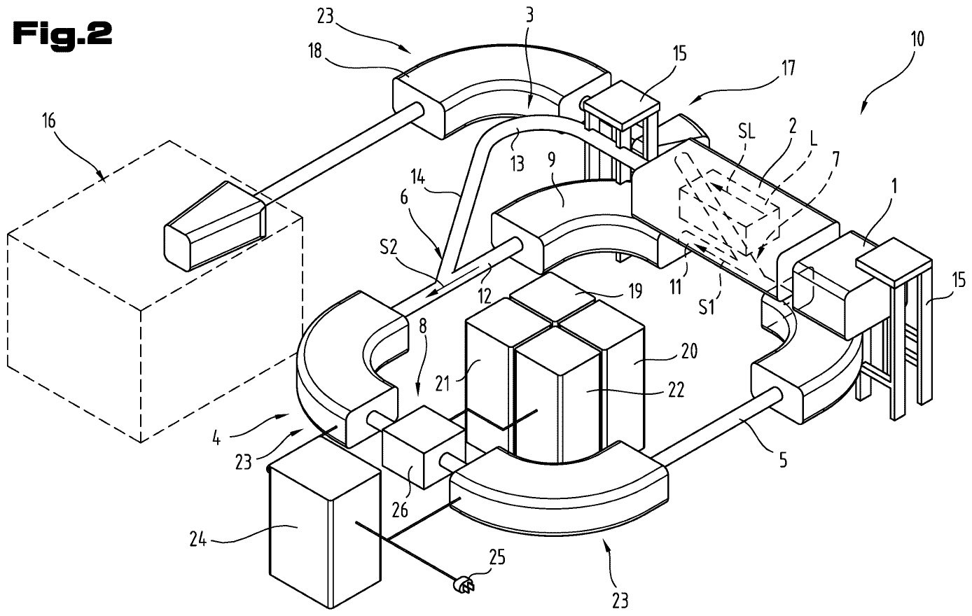

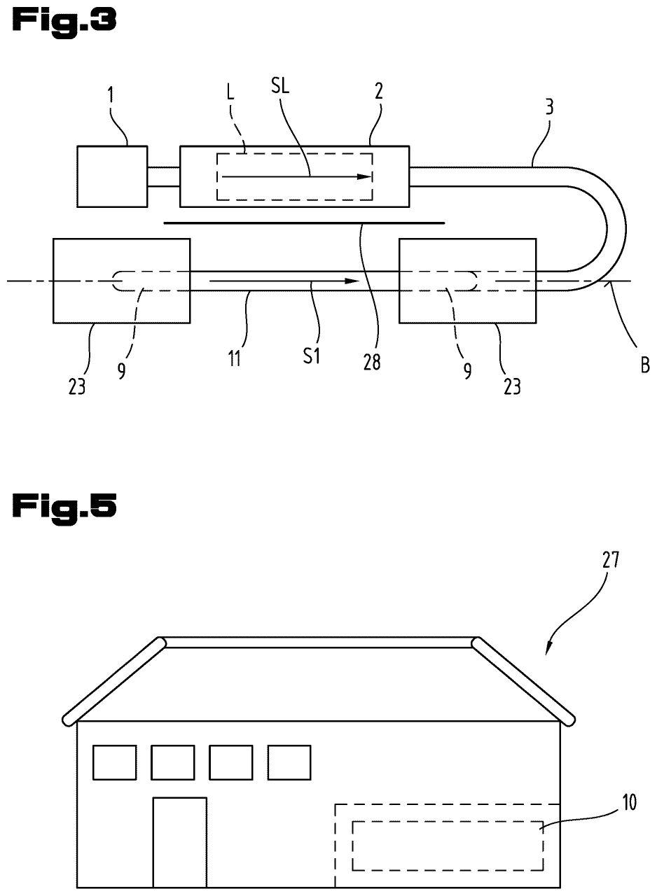

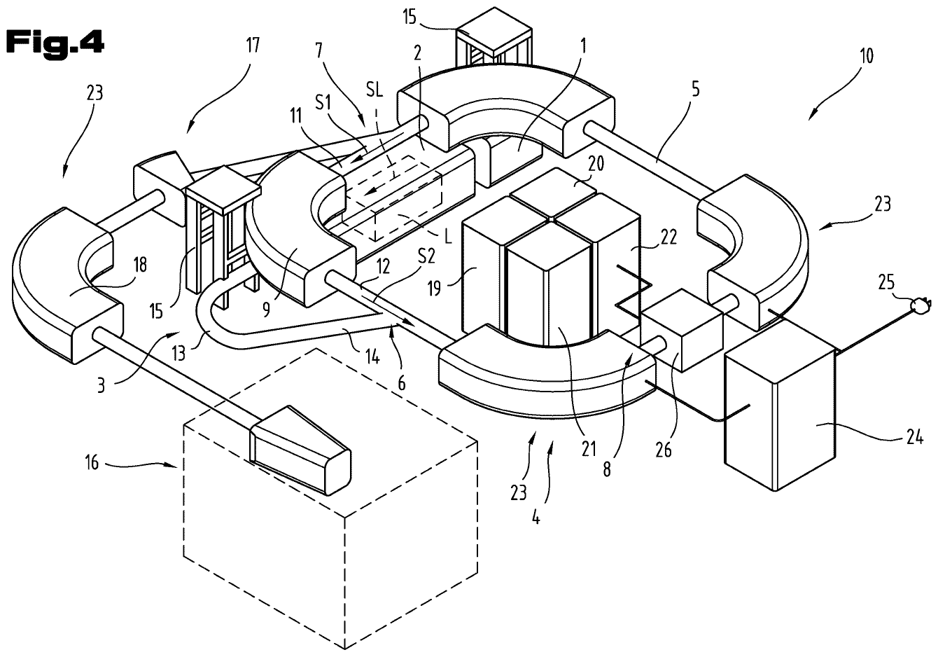

According to the disclosure, the pre-acceleration device 2 is arranged above or below the path plane B of the synchrotron 4 (see also FIGS. 3 and 4), whereby the pre-acceleration device 2 vertically overlaps a first path section 11 of the circulating particle path 5 of the synchrotron 4 (see FIGS. 1 and 2). The pre-acceleration device 2 can overlap vertically with the first path section 11 along at least 50%, preferably at least 75%, and particularly preferably at least 100% of its longitudinal extent or the longitudinal extent of its beam path. In the embodiment shown, the overlap occurs continuously along almost the entire length of the pre-acceleration device 2.

The pre-acceleration device 2 can be arranged vertically above (FIG. 1) or vertically below (FIG. 4) the first path section 11.

FIGS. 1 and 2 show that the extraction site 7 can be arranged in the first path section 11 and that the pre-acceleration device 2 can overlap vertically with the extraction site 7. The particle source 1 can also overlap vertically with the first path section 11 of the circulating particle path 5 of the synchrotron 4.

In the preferred embodiment shown in FIG. 1, the first path section 11 is a linear path section and the pre-acceleration device 2 comprises a linear accelerator L. The beam path S1 of the first path section 11 and the beam path SL of the linear accelerator L are essentially parallel to each other. Alternatively, the beam paths S1 and SL may be inclined relative to each other, preferably by an angle of no more than 20° (i.e., with a deviation from a parallel alignment of no more than 20°).

The transfer beam guide 3 overcomes a difference in height between the pre-acceleration device 2 and the injection site 6. The deflection is achieved using appropriately dimensioned magnets. In the embodiments shown, the transfer beam guide 3 extends laterally beyond the outer contour of the circulating particle path 5 of the synchrotron 4.

The transfer beam guide 3 preferably enters the injection site 6 with a horizontal directional component, preferably from a region outside the region enclosed by the particle path 5 (see FIGS. 1 and 4).

The transfer beam guide 3 may have a first path section 13, which is used to overcome the height difference between the pre-acceleration device 2 and the path plane B of the synchrotron 4, and a second path section 12, which runs between the first path section 11 and the injection site 6 in the path plane B of the synchrotron 4.

As can be seen in the embodiments of FIGS. 1 and 4, the injection site 6 may be arranged in a second, preferably linear, path section 12 of the synchrotron 4. Here, the beam direction S2 defined by the second path section 12 and the beam direction S1 defined by the first path section 11 are inclined to each other by an angle that is preferably at least 60° and no more than 120°, particularly preferably 90°. The first path section 11, which is a linear path section, transitions directly into an arcuate path section 9, and the arcuate path section 9 transitions directly into the second path section 12, which is a linear path section. In the embodiment shown, the arcuate path section 9 is a 90° arc.

FIG. 2 shows that an electromagnetic shield 8, preferably in the form of a plate, foil, grid, or mesh, made of electrically conductive material, can be arranged between the pre-accelerator device 2 and the first path section 11 in order to eliminate mutual interference between the components arranged above each other. It is also preferable if the pre-accelerator device 2 is at least partially supported by the same frame that also supports the synchrotron 4.

The pre-accelerator device 2 and/or the particle source 1 may be supported by at least one, preferably at least two, support strut(s) 15 extending from bottom to top. In order to make optimum use of the available space, at least one support 15 is arranged on the outside of an arcuate path section 9 of the synchrotron 4 (i.e., in the corner region(s) of an (imaginary) rectangular contour closely enclosing the synchrotron 4 that is/are left free by the arcuate path section(s)).

As can be seen in the figures, the particle system 10 comprises an extraction beam guide 17 extending from the extraction site 7 and leading to a treatment unit 16. This may have an arcuate path section 18 (here: in the form of a 90° arc). The particular arrangement of the individual components in the preferred embodiment shown indicates that optimum utilization of the available space can be achieved by ensuring that the projection of the transfer beam guide 3 into the path plane of the synchrotron 4—at least partially (preferably entirely)—lies in a region between the particle path 5 of the synchrotron 4 and the extraction beam guide 17.

These measures make it possible to achieve, and such an embodiment is particularly preferred, that an (imaginary) contour drawn around the arrangement of synchrotron 4, particle source 1, pre-acceleration device 2, extraction beam guide 17, and treatment unit 16 in a horizontal plane encloses an area of no more than 100 m², preferably a maximum area of 85 m².

FIGS. 1, 2, and 4 also show that the particle therapy system 10 has a power supply device 19 (in particular for supplying the particle or ion source, the pre-acceleration device, the cavity or cavities, the (deflection) magnets, etc.), a vacuum generation device 20 (for generating a vacuum in the tube forming the particle path), a cooling device 21 (for cooling the aforementioned components) and a control device 22 (for controlling the process). For optimal use of space, at least one, preferably at least two, of these components is/are arranged above and/or below the synchrotron 4 and/or within the circulating particle path 5 of the synchrotron 4. In the embodiment shown, all of these components are arranged in the interior region enclosed by the ring accelerator.

Finally, it should be noted that the embodiments shown in the figures and described above represent preferred embodiments of the disclosure. Of course, it is conceivable that the pre-acceleration device is arranged above or below another path section of the synchrotron, as long as it overlaps vertically with this path section. It is also not absolutely necessary for the extraction site to be formed in the first path section. The injection can also take place at another point. The transfer beam path can be dimensioned accordingly to be longer or shorter. Many other variants are possible.

In combination with or independently of the features described above, the particle therapy system 10 may have a control device 22 with an operating mode for feeding the energy stored in the magnets 23—provided for guiding or deflecting the particles—back into an electrical energy storage device 24 or into the power grid 25.

Similarly, in combination with or independently of the features described above, the particle therapy system 10 may comprise a control device 22 with an operating mode for decelerating the particles in the synchrotron 4, wherein the operating mode is configured to reduce the velocity of the particles by controlling at least one cavity 26 of an acceleration section 8 of the synchrotron 4, in that the at least one cavity 26 generates a force acting on the particles against their direction of movement. This can be achieved by applying the high-frequency coil(s) of the cavity 26 in such a way that the high-frequency electromagnetic waves accelerate the particles against their direction of movement (i.e., actively decelerate them).

Finally, FIG. 5 schematically shows a hospital 27 containing a particle therapy system 10 according to the disclosure. As already mentioned at the outset, the significantly reduced space requirement makes it possible to install such a particle therapy system in smaller rooms and thus also in hospital premises.

Finally, the disclosure also relates to a method for operating a particle therapy system 10, in which method the particles coming from the particle source 1 are accelerated by the pre-acceleration device 2 and injected into the synchrotron 4 by means of the transfer beam guide 3.

In addition, in one operating mode of the particle therapy system 10, the energy stored in the magnets 23—provided for guiding or deflecting the particles—can be fed into an electrical energy storage device 24 or into the power grid 25.

In a further operating mode, the particles in the synchrotron 4 can be decelerated (actively, i.e., by introducing electromagnetic energy) by controlling at least one cavity 26 of an acceleration section 8 of the synchrotron 4 in order to reduce the speed of the particles by generating force acting on the particles in the opposite direction to their direction of movement through the at least one cavity 26.

### LIST OF REFERENCE SYMBOLS

1 particle source
2 pre-acceleration
3 transfer beam guide
4 synchrotron
5 circulating particle path
6 injection site
7 extraction site
8 acceleration section
9 arcuate path section of the particle path 5
10 particle therapy system
11 first section of the circulating particle path 5
12 second section of the circulating particle path 5
13 first section of the transfer beam guide 3
14 second section of the transfer beam guide 3
15 support strut
16 treatment unit
17 extraction beam guide
18 arcuate path section of the extraction beam guide 17
19 energy supply device
20 vacuum generation device
21 cooling device
22 control device
23 magnets
24 electrical energy storage device
25 power grid
26 cavity
27 hospital
28 shield
B path plane
L linear accelerator
S1 beam path of the first path section 11
S2 beam path of the second path section 12
SL beam path of the linear accelerator L

1. A particle therapy system in particular a hadron therapy system, comprising:
a particle source,
a pre-acceleration device connected to the particle source for accelerating the particles coming from the particle source,
a synchrotron with a circulating particle path having an injection site and an extraction site, and
a transfer beam guide leading from the pre-acceleration device to the injection site for injecting the particles accelerated by the pre-acceleration device into the particle path of the synchrotron,
wherein the pre-acceleration device is arranged above or below the path plane of the synchrotron, wherein the pre-acceleration device overlaps, at least partially, vertically with a first path section of the circulating particle path of the synchrotron.

2. The particle therapy system according to claim 1, wherein the pre-acceleration device is arranged vertically above or vertically below the first path section.

3. The particle therapy system according to claim 1, wherein the extraction site is arranged in the first path section and the pre-acceleration device vertically overlaps the extraction site.

4. The particle therapy system according to claim 1, wherein the particle source vertically overlaps the first path section of the circulating particle path of the synchrotron.

5. The particle therapy system according to claim 1, wherein the first path section is a linear path section and the pre-acceleration device comprises a linear accelerator, wherein the beam path of the first path section and the beam path of the linear accelerator are essentially parallel to each other or have a deviation from a parallel alignment of no more than 20°.

6. The particle therapy system according to claim 1, wherein the transfer beam guide between the pre-acceleration device and the injection site overcomes a difference in height and/or that the transfer beam guide extends laterally beyond the outer contour of the circulating particle path of the synchrotron.

7. The particle therapy system according to claim 1, wherein the transfer beam guide has a first path section with which the height difference between the pre-acceleration device and the path plane of the synchrotron is overcome, and a second path section which runs between the first path section and the injection site in the path plane of the synchrotron.

8. The particle therapy system according to claim 1, wherein the transfer beam guide opens into the injection site with a horizontal directional component, preferably from a region outside the region enclosed by the particle path.

9. The particle therapy system according to claim 1, wherein the injection site is arranged in a second, preferably linear, path section of the synchrotron, wherein the beam direction defined by the second path section and the beam direction defined by the first path section are inclined to each other by an angle which is preferably at least 60° and no more than 120°.

10. The particle therapy system according to claim 1, wherein the first path section which is a linear path section, transitions directly into an arcuate path section, and the arcuate path section transitions directly into the second path section, which is a linear path section, wherein the arcuate path section is preferably a 90° arc.

11. The particle therapy system according to claim 1, wherein an electromagnetic shield, preferably in the form of a plate, foil, grid, or mesh, made of electrically conductive material, is arranged between the pre-accelerator device and the first path section.

12. The particle therapy system according to claim 1, wherein the pre-accelerator device is at least partially supported by the same frame that also supports the synchrotron.

13. The particle therapy system according to claim 1, wherein the pre-accelerator device and/or the particle source is supported by at least one, support strut extending from bottom to top, at least one support strut being arranged on the outside of an arcuate path section of the synchrotron.

14. The particle therapy system according to claim 1, wherein the particle system comprises an extraction beam guide extending from the extraction site and leading to a treatment unit, wherein the extraction beam guide preferably has an arcuate path section.

15. The particle therapy system according to claim 14, wherein the projection of the transfer beam guide into the path plane of the synchrotron lies in a region between the particle path of the synchrotron and the extraction beam guide.

16. The particle therapy system according to claim 1, wherein a contour drawn in a horizontal plane around the arrangement consisting of the synchrotron, particle source, pre-acceleration device, extraction beam guide and treatment unit in a horizontal plane encloses an area of no more than 100 m², preferably no more than 85 m².

17. The particle therapy system according to claim 1, wherein the particle therapy system comprises an energy supply device, a vacuum generation device, a cooling device and a control device, wherein at least one of these components is arranged above and/or below the synchrotron and/or within the circulating particle path of the synchrotron.

18. The particle therapy system according to claim 1, wherein the pre-acceleration device vertically overlaps the first path section along at least 50% of its longitudinal extension or the longitudinal extension of its beam path.

19. The particle therapy system according to claim 1, wherein the first path section is an exclusively linear path section and that the ratio between the length of the beam path within the pre-acceleration device or within a linear accelerator of the pre-acceleration device and the length of the first path section is between 0.7 and 1.3.

20. A particle therapy system, in particular a hadron therapy system, comprising:
a particle source,
a pre-acceleration device connected to the particle source for accelerating the particles coming from the particle source,
a synchrotron with a circulating particle path having an injection site and an extraction site, and
a transfer beam guide leading from the pre-acceleration device to the injection site for injecting the particles accelerated by the pre-acceleration device into the particle path of the synchrotron,
wherein the particle therapy system comprises a control device with an operating mode that is operable to perform at least one of:
feed energy stored in magnets or into a power grid,
decelerate particles in the synchrotron, wherein the operating mode is configured to reduce the velocity of the particles by controlling at least one cavity of an acceleration section of the synchrotron to reduce the velocity of the particles by generating a force acting on the particles in the at least one cavity in the opposite direction to their direction of movement.

21. (canceled)

22. The particle therapy system as claimed in claim 20, characterized in that a particle therapy system is arranged in a hospital.

23. A method for operating a particle therapy system wherein particles coming from a particle source are accelerated by a pre-acceleration device and are injected into a synchrotron by means of a transfer beam guide.

24. The method for operating a particle therapy system according to claim 23, wherein the particle therapy system comprises:
a particle source,
a pre-acceleration device connected to the particle source for accelerating the particles coming from the particle source,
a synchrotron with a circulating particle path having an injection site and an extraction site, and
a transfer beam guide leading from the pre-acceleration device to the injection site for injecting the particles accelerated by the pre-acceleration device into the particle path of the synchrotron,
wherein in one operating mode of the particle therapy system, energy stored in magnets is fed into an electrical energy storage device or into a power grid.

25. The method for operating a particle therapy system according to claim 23, wherein the particle therapy system comprises:
a particle source,
a pre-acceleration device connected to the particle source for accelerating particles coming from the particle source,
a synchrotron with a circulating particle path having an injection site and an extraction site, and
a transfer beam guide leading from the pre-acceleration device to the injection site for injecting the particles accelerated by the pre-acceleration device into the particle path of the synchrotron,
wherein in one operating mode, the particles in the synchrotron are decelerated by controlling at least one cavity of an acceleration section of the synchrotron in order to reduce the velocity of the particles by generating a force acting on the particles through the at least one cavity in the opposite direction to their direction of movement.
